(19) United States
(12) Patent Application Publication
(10) Pub. No.: US 2018/0267065 A1
(43) Pub. Date: Sep. 20, 2018

(54) METHODS RELATING TO THE MEASUREMENT OF VITAMIN D AND VITAMIN D METABOLITES

(71) Applicant: Trustees of Boston University, Boston, MA (US)

(72) Inventor: Michael Francis Holick, Sudbury, MA (US)

(73) Assignee: Trustees of Boston University, Boston, MA (US)

(21) Appl. No.: 15/921,909

(22) Filed: Mar. 15, 2018

Related U.S. Application Data
(60) Provisional application No. 62/473,641, filed on Mar. 20, 2017.

(51) Int. Cl. G01N 33/82; G01N 1/44

(52) U.S. Cl. CPC G01N 33/82; G01N 1/44

(57) ABSTRACT

Described herein are methods for measuring analytes, wherein a portion of the analyte in a sample is bound by a binding protein. In some embodiments, the methods relate to measuring vitamin D or metabolites thereof, e.g., vitamin D in samples also comprising a vitamin D binding protein.

### CROSS-REFERENCE TO RELATED APPLICATIONS

This application claims benefit under 35 U.S.C. § 119(e) of U.S. Provisional Application No. 62/473,641 filed Mar. 20, 2017, the contents of which are incorporated herein by reference in their entirety.

### TECHNICAL FIELD

The technology described herein relates to the measurement of compounds in a sample, e.g., vitamin D and vitamin D metabolites.

### BACKGROUND

The measurement of certain compounds in biological samples is complicated by the fact that the compounds can be bound by proteins found in the sample, rendering the bound compounds unavailable for analysis. This bound fraction causes current methods to underestimate the level of the compound and/or to increase the noise in the measurments of the sample.

### SUMMARY

As described herein, the inventors have found that heating the samples containing compounds to be measured can denature the relevant binding proteins, thereby ensuring the bound fraction of the compound is eliminated or significantly reduced, e.g., by causing the bound fraction to be freed from protein binding. Such heat denaturation is readily adapted to automated platforms and demonstrates improved efficacy over denaturation with, e.g., an acid or base.

In one aspect of any of the embodiments, described herein is a method of measuring the level of an analyte in a sample wherein a portion of the analyte in the sample is bound by at least one binding protein, the method comprising: heating the sample to a temperature at which the binding protein is denatured; and measuring the level of the analyte. In some embodiments of any of the aspects, the analyte is one or more of: 25-hydroxyvitamin D3; 25-hydroxyvitamin D2; 1,25-dihydroxyvitamin D3; 1,25-dihydroxyvitamin D2; vitamin D3; vitamin D2 or a metabolite thereof.

In some embodiments of any of the aspects, the temperature is at least 40° C. In some embodiments of any of the aspects, the temperature is from 40° C. to 250° C. In some embodiments of any of the aspects, the temperature is at least 200° C. In some embodiments of any of the aspects, the heating step comprises exposing the sample to at least one of: an electric heating device; an infrared signal; an electromagnetic signal; a microwave signal; or an exothermic chemical reaction.

In some embodiments of any of the aspects, the method further comprises a step after the heating step and before the measuring step of extracting the sample. In some embodiments of any of the aspects, the method further comprises a step after the heating step and before the measuring step of extracting the sample with an organic solvent. In some embodiments of any of the aspects, the measuring step comprises a chemiluminescence assay or radioimmunoassay. In some embodiments of any of the aspects, the measuring step comprises liquid chromatography. In some embodiments of any of the aspects, the measuring step comprises mass spectrometry. In some embodiments of any of the aspects, the measuring step comprises measuring the level of vitamin D and/or a metabolite thereof.

In some embodiments of any of the aspects, the sample is a serum or plasma sample. In some embodiments of any of the aspects, the sample is a saliva sample.

In some embodiments of any of the aspects, the pH of the sample is no lower than 5.6 during the method.

### DETAILED DESCRIPTION

As described herein, the inventors have found that heat denaturation of the binding proteins in a sample releases compounds bound by those proteins, making the compounds more readily available for analysis (e.g. measurement of the total level of the compound in a sample). Surpisingly, the standard approach of acid or base denaturation does not completely release all bound vitamin D and vitamin D metabolites especially vitamin D2 metabolites and specifically 25-hydroxyvitamin D2. In view of the fact that vitamin D is heat stable up to at least 200° C., the inventor has found that heating the sample for a very brief period of time will rapidly denature all proteins present in the sample by destroying the secondary and tertiary protein structures and releasing any bound vitamin D and vitamin D. This makes available all vitamin D and vitamin D metabolites for extraction, e.g., with an organic solvent.

The methods described herein relate the measurement of the level and/or other characteristic of a compound or analyte. As used herein, “analyte” refers to any entity, compound, structure, or molecule which can be bound by a protein. In some embodiments of any of the aspects, the analyte can be bound by a protein found in a biological sample. In some embodiments of any of the aspects, the analyte is an analyte which naturally occurs in a biological sample with at least a portion of the analyte being bound by a naturally-occuring protein in that sample. Exemplary analytes can include ions, proteins, peptides, organic compounds, steroids, inorganic compounds, or the like. In some embodiments of any of the aspects, the analyte is a non-proteinaceous organic compound. In some embodiments of any of the aspects, the analyte is a steroid. In some embodiments of any of the aspects, the analyte is a vitamin D-related compound or metabolite thereof.

As used herein “vitamin D” or “calciferol” is a general name that refers to a collection of steroid-like substances including vitamin D₂ (ergocalciferol) and vitamin D₃ (cholecalciferol).

Vitamin D₃ is a secosteroid. The IUPAC name is (3β,5Z,7E)-9,10-secocholesta-5,7,10(19)-trien-3-ol. The chemical structure of vitamin D₃ is as follows:

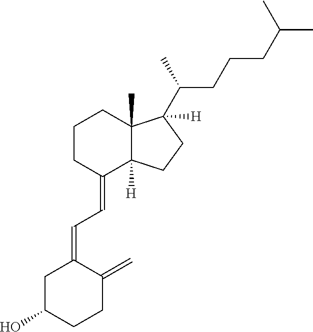 [SMILES: C=C1CC[C@H](O)C/C1=C/C=C1\CCC[C@]2(C)[C@@H]([C@H](C)CCCC(C)C)CC[C@@H]12]

Vitamin D₃ is metabolized by the liver to 25(OH)D₃ (also known as 25 hydroxycholecalciferol, calcifediol, or calcidiol), which is then converted by the kidneys to 1,25(OH)₂D₃ (also known as 1,25 dihydroxycholecalciferol, calcitriol, or active vitamin D hormone). 25(OH)D₃, the major circulating form, has some metabolic activity, but 1,25(OH)₂D₃ is the most metabolically active.

The chemical structure of 25(OH)D₃ is as follows:

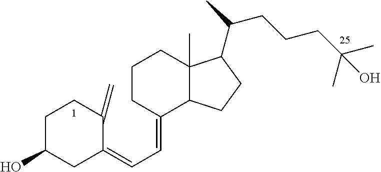

The chemical structure of 1,25(OH)₂D₃ is as follows:

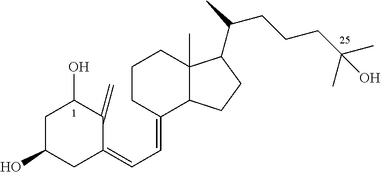

Vitamin D₂ is a secosteroid. The IUPAC name is (3β,5Z,7E,22E)-9,10-secoergosta-5,7,10(19),22-tetraen-3-ol. The chemical structure of vitamin D₂ is as follows:

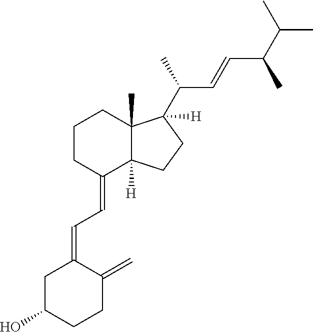 [SMILES: C=C1CC[C@H](O)C/C1=C/C=C1\CCC[C@]2(C)[C@@H]([C@H](C)/C=C/[C@H](C)C(C)C)CC[C@@H]12]

Similarly to vitamin D₃, vitamin D₂ is metabolized to 25-hydroxyvitamin D₂ and 1,25-dihydroxyvitamin D₂.

As used herein, vitamin D-related compound refers to any of the foregoing vitamin D's and their metabolites. Exemplary vitamin D-related compounds can include 25-hydroxyvitamin D₃; 25-hydroxyvitamin D₂; 1,25-dihydroxyvitamin D₃; 1,25-dihydroxyvitamin D₂; vitamin D₃; and/or vitamin D₂.

Vitamin D-related compounds can be bound by, e.g., vitamin D binding protein (e.g., NCBI Gene ID: 2638) or the vitamin D receptor (e.g., NCBI Gene ID: 7421) as well as albumin and lipoproteins. For more discussion, see, e.g., Haddad et al. J CI 1993. 91:2552-2555; which is incorporated by reference herein in its entirety. In some embodiments, a protein that binds vitamin D and/or vitamin D-related compounds can be vitamin D binding protein; the vitamin D receptor; and/or variants, fragments, or homologs thereof as well as albumin and lipoproteins.

In one aspect of any of the embodiments, described herein is a method of measuring the level of an analyte in a sample wherein a portion of the analyte in the sample is bound by at least one binding protein, the method comprising: heating the sample to a temperature at which the binding protein is denatured; and measuring the level of the analyte. In some embodiments of any of the aspects, the analyte is one or more vitamin D-related compounds and/or metabolites thereof. While proteins can be denatured by a a number of means, heat denaturation as described herein is preferred.

The temperature at which a given binding protein denatures will vary with the characteristics of the solution and/or sample, e.g., the buffer concentration, pH, etc., and is readily determined by one of skill in the art.

In some embodiments of any of the aspects, the temperature at which the binding protein is denatured is at least about 40° C. In some embodiments of any of the aspects, the temperature at which the binding protein is denatured is at least about 40° C., eg., at least about 50° C., at least about 60° C., at least about 70° C., at least about 80° C., at least about 90° C., at least about 100° C., at least about 110° C., at least about 125° C., at least about 150° C., at least about 175° C., at least about 200° C., or greater. In some embodiments of any of the aspects, the temperature at which the binding protein is denatured is at least 40° C. In some embodiments of any of the aspects, the temperature at which the binding protein is denatured is at least 40° C., eg., at least 50° C., at least 60° C., at least 70° C., at least 80° C., at least 90° C., at least 100° C., at least 110° C., at least 125° C., at least 150° C., at least 1750 C, at least 200° C., or greater.

In some embodiments of any of the aspects, the temperature at which the binding protein is denatured is at least about 190° C. In some embodiments of any of the aspects, the temperature at which the binding protein is denatured is at least about 200° C. In some embodiments of any of the aspects, the temperature at which the binding protein is denatured is at least about 210° C. In some embodiments of any of the aspects, the temperature at which the binding protein is denatured is at least 190° C. In some embodiments of any of the aspects, the temperature at which the binding protein is denatured is at least 200° C. In some embodiments of any of the aspects, the temperature at which the binding protein is denatured is at least 210° C.

In some embodiments of any of the aspects, the temperature at which the binding protein is denatured is from about 40° C. to about 250° C. In some embodiments of any of the aspects, the temperature at which the binding protein is denatured is from 40° C. to 250° C. In some embodiments of any of the aspects, the temperature at which the binding protein is denatured is from about 50° C. to about 250° C. In some embodiments of any of the aspects, the temperature at which the binding protein is denatured is from 50° C. to 250° C. In some embodiments of any of the aspects, the temperature at which the binding protein is denatured is from about 60° C. to about 250° C. In some embodiments of any of the aspects, the temperature at which the binding protein is denatured is from 60° C. to 250° C. In some embodiments of any of the aspects, the temperature at which the binding protein is denatured is from about 75° C. to about 250° C. In some embodiments of any of the aspects, the temperature at which the binding protein is denatured is from 75° C. to 250° C. In some embodiments of any of the aspects, the temperature at which the binding protein is denatured is from about 100° C. to about 250° C. In some embodiments of any of the aspects, the temperature at which the binding protein is denatured is from 100° C. to 250° C. In some embodiments of any of the aspects, the temperature at which the binding protein is denatured is from about 150° C. to about 250° C. In some embodiments of any of the aspects, the temperature at which the binding protein is denatured is from 150° C. to 250° C. In some embodiments of any of the aspects, the temperature at which the binding protein is denatured is from about 200° C. to about 250° C. In some embodiments of any of the aspects, the temperature at which the binding protein is denatured is from 200° C. to 250° C.

Heat can be applied to the sample by any one of, or a combination of various methods. Suitable methods include, e.g., exposing the sample to an electric heating device; exposing the sample to an infrared or microwave radiation; exposing the sample to an exothermic chemical reaction; and/or exposing the sample to radiation, e.g., electromagnetic radiation, e.g., electromagnetic radiation that increases the temperature of the sample to the desired temperature (e.g., microwave and/or radar radiation). Additional heat sources can include RF heating, water baths, microwave heating, a resistive heater, a radiative heat source, and the like. The exposure can be direct, e.g, the sample is directly contacted with the heat source and/or the heat source is placed in or submerged in the sample. The exposure can also be indirect, e.g, the heat is transferred through a container holding the sample and optionally, through one or more additional substances, e.g., air, a heat conductive material, etc.

Exposing the sample to the heat source can comprise placing the sample in a device specifically configured for that purpose (e.g., Digi-Block™ Cat. No. Barnstead International 5035 from Sigma-Aldrich, St. Louis Mo.) or incorporating and/or using a device, unit, protocol, or functionality in a device which performs analyte measurement functions (e.g. the Cobas™ series from Roche Diagnostics or the ARCHITECT™ series instruments from Abbott).

In some embodiments of any of the aspects, the method can further comprise a step after the heating step and before the measuring step of extracting the sample, e.g., extracting the analyte and/or a portion of the sample comprising the analyte from the starting sample and/or extracting proteins from the sample. In some embodiments, the extracting of the sample can comprise extracting the sample with an organic solvent. In some embodiments, the extracting step can comprise separating the sample into at least a first portion comprising the analyte and a second portion comprising proteins which can bind the analyte. Methods of extracting various types of proteins, analytes and/or specific analytes are known to those of skill in the art. By way of non-limiting example, vitamin D and/or vitamin D-related compounds can be extacted using acetonitrile, EX Reagent™ (Diazyme); the Vitamin D extraction kit from IBL-America (Cat. No. 3019700; Minneapolis, Minn.); and the like.

The measurement step can comprise any method known in the art for detecting a characteristic of the analyte, e.g., the level of the analyte in the sample. Suitable types of measurements will vary depending upon the identity of the analyte, the level of accuracy and/or rapidity of measurement required, the characteristic to be measured, sample volume, number of samples and the like. One of skill in the art can readily select a suitable measurement technique for a given set or subset of the foregoing parameters. By way of non-limiting example, suitable measurement steps and/or assays can include a chemiluminescence assay, radioimmunoassay, immunoassay, mass spectrometry; Western blot; immunoprecipitation; enzyme-linked immunosorbent assay (ELISA); radioimmunological assay (MA); sandwich assay; fluorescence in situ hybridization (FISH); immunohistological staining; lateral flow immunoassay (LFIA); radioimmunometric assay; immunofluoresence assay; and/or immunoelectrophoresis assay.

Immunochemical methods require the use of an antibody reagent specific for the target molecule (e.g., the analyte). In some embodiments, the antibody reagent can be attached to a solid support (e.g., bound to a solid support). In some embodiments, the solid support can comprise a particle (including, but not limited to an agarose or latex bead or particle or a magnetic particle), a bead, a nanoparticle, a polymer, a substrate, a slide, a coverslip, a plate, a dish, a well, a membrane, and/or a grating. The solid support can include many different materials including, but not limited to, polymers, plastics, resins, polysaccharides, silicon or silica based materials, carbon, metals, inorganic glasses, and membranes.

In some embodiments, the antibody reagent can be detectably labeled. Detectable labels can comprise, for example, a light-absorbing dye, a fluorescent dye, or a radioactive label. Detectable labels, methods of detecting them, and methods of incorporating them into an antibody reagent are well known in the art. In some embodiments, detectable labels can include labels that can be detected by spectroscopic, photochemical, biochemical, immunochemical, electromagnetic, radiochemical, or chemical means, such as fluorescence, chemifluoresence, or chemiluminescence, or any other appropriate means. The detectable labels used in the methods described herein can be primary labels (where the label comprises a moiety that is directly detectable or that produces a directly detectable moiety) or secondary labels (where the detectable label binds to another moiety to produce a detectable signal, e.g., as is common in immunological labeling using secondary and tertiary antibodies). The detectable label can be linked by covalent or non-covalent means to the antibody reagent. Alternatively, a detectable label can be linked such as by directly labeling a molecule that achieves binding to the antibody reagent via a ligand-receptor binding pair arrangement or other such specific recognition molecules. Detectable labels can include, but are not limited to radioisotopes, bioluminescent compounds, chromophores, antibodies, chemiluminescent compounds, fluorescent compounds, metal chelates, and enzymes.

In some embodiments of any of the aspects, the measuring step comprises measuring the level of a vitamin D-related compound, vitamin D and/or a metabolite thereof. Suitable means of conducting the measurement step and/or suitable assays for vitamin D measurements are known in the art and can include, by way of non-limiting example, the Siemens ADVIA Centaur assay (Erlangen, Germany); the Roche ELECSYS™ assay (Rotkreuz, Switzerland); the DiaSorin LIAISON™ assay (Cat No. 310600; Saluggia, Italy); the Abbott ARCHITECT assay (Chicago, Ill.); ClinMass® LC-MS/MS Complete Kit (RECIPE Chemicals+Instruments GmbH, Munich, Germany); the ORGENTEC 25(OH)D3/D2 ELISA (ORGENTEC Diagnostika GmbH, Mainz, Germany); Immunodiagnostic Systems (IDS)-iSYS 25(OH)DS and IDS-iSYS 25(OH)D CLIA (Immunodiagnostic Systems Ltd, Boldon, United Kingdom); the Diazyme Laboratories' 25-OH Vitamin D Assay (Poway, Calif.); and the Vitamin D ELISA Kit (Item No 501050; Cayman Chemicals, Ann Arbor, Mich.).

The term “sample” or “test sample” as used herein denotes a sample taken or isolated from an organism, e.g., a urine sample from a subject. Exemplary biological samples include, but are not limited to, a biofluid sample; serum; plasma; urine; saliva etc. The term also includes a mixture of the above-mentioned samples. The term “test sample” also includes untreated or pretreated (or pre-processed) biological samples. In some embodiments, a test sample can comprise cells from a subject. As used herein, the term “biofluid” refers to any fluid obtained from a biological source and includes, but is not limited to, blood, urine, and bodily secretions.

The test sample can be obtained by removing a sample from a subject, but can also be accomplished by using a previously isolated sample (e.g., isolated at a prior timepoint and isolated by the same or another person). In addition, the test sample can be freshly collected or a previously collected sample.

In some embodiments, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments, the test sample can be a frozen test sample, e.g., a frozen tissue. The frozen sample can be thawed before employing methods, assays and systems described herein. After thawing, a frozen sample can be centrifuged before being subjected to methods, assays and systems described herein. In some embodiments, the test sample is a clarified test sample, for example, prepared by centrifugation and collection of a supernatant comprising the clarified test sample. In some embodiments, a test sample can be a pre-processed test sample, for example, supernatant or filtrate resulting from a treatment selected from the group consisting of centrifugation, filtration, thawing, purification, and any combinations thereof. In some embodiments, the test sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. The skilled artisan is well aware of methods and processes appropriate for pre-processing of biological samples required for determination of the level of an analyte as described herein.

In some embodiments of any of the aspects, the sample is a serum sample, plasma sample, and/or saliva sample.

In some embodiments of any of the aspects, the methods described herein can further comprise a step of obtaining a test sample from a subject. In some embodiments of any of the aspects, the subject can be a human subject.

In some embodiments of any of the aspects, the methods described herein can relate to methods that do not comprise exposing the sample to acidic and/or basic conditions, e.g., to denature the proteins. In some embodiments of any of the aspects, the pH of the sample is no lower than 6.5 during the method. In some embodiments of any of the aspects, the pH of the sample is no lower than 6.0 during the method. In some embodiments of any of the aspects, the pH of the sample is no lower than 5.6 during the method. In some embodiments of any of the aspects, the pH of the sample is no greater than 7.5 during the method. In some embodiments of any of the aspects, the pH of the sample is no greater than 8.0 during the method. In some embodiments of any of the aspects, the pH of the sample is no greater than 8.5 during the method.

In some embodiments, the methods, assays, and systems described herein can comprise creating a report based on the level of the analyte. In some embodiments, the report denotes raw values for the analyte in the test sample (plus, optionally, the level of the analyte in a reference sample) or it indicates a percentage or fold increase in the analyte as compared to a reference level.

Vitamin D deficiency can contribute to a number of pathologies. Accurate identification of vitamin D deficiency, e.g, by accurate measurement of vitamin D levels is key to treating vitamin D deficiency as well as treating and/or preventing other conditions. The assays and methods as described herein can relate to determining if a subject has decreased level of a vitamin D-related compound relative to a reference level. The methods and assays described herein can relate to a method of detecting, prognosing, and/or diagnosing a vitamin D deficiency, the method comprising detecting or measuring the level of a vitamin D-related compound(s) in a sample obtained from a subject as described herein, wherein a decrease in the vitamin D-related compound(s) indicates the subject has a vitamin D deficiency. The methods and assays described herein can relate to a method of identifying a subject in need of treatment for vitamin D deficiency, the method comprising detecting or measuring the level of a vitamin D-related compound(s) in a sample obtained from a subject as described herein, wherein a decrease in the vitamin D-related compound(s) indicates the subject is in need of treatment for vitamin D deficiency. The methods and assays described herein can relate to a method of treating a subject with a vitamin D deficiency, the method comprising detecting or measuring the level of a vitamin D-related compound(s) in a sample obtained from a subject as described herein, and administering a treatment (e.g., vitamin D) when a decrease in the vitamin D-related compound(s) is detected.

In some embodiments, the reference level of a vitamin D-related compound(s) can be the level of vitamin D-related compound(s) in a healthy subject not having, or not diagnosed as having, e.g., vitamin D deficiency. In some embodiments, the reference level can be the level in a sample of similar cell type, sample type, sample processing, and/or obtained from a subject of similar age, sex and other demographic parameters as the sample/subject for which the level of the vitamin D-related compound(s) is to be determined. In some embodiments, the test sample and control reference sample are of the same type, that is, obtained from the same biological source, and comprising the same composition, e.g., the same number and type of cells and/or type of sample material. Accordingly, in some embodiments, a level of a vitamin D-related compound(s) which is increased can vary as demographic factors such as age, gender, genotype, environmental factors, and individual medical histories vary. In some embodiments of any of the aspects, the reference can be a level in a control subject, in a control sample, a pooled sample of control individuals or a numeric value or range of values based on the same. In some embodiments, the reference level of a vitamin D-related compound(s) can be the level of the vitamin D-related compound(s) in a prior sample obtained from the subject. This permits a direct analysis of any change in levels in that individual.

In some embodiments, a level of a vitamin D-related compound(s) can be decreased relative to a reference level if the level of the vitamin D-related compound(s) is decreased by a statistically significant amount. In some embodiments, a level of a vitamin D-related compound(s) can be decreased relative to a reference level if the level of the vitamin D-related compound(s) is decreased by 1 σ, e.g., by 1 σ, 1.5 σ, 2 σ, or more. In some embodiments, a level of a vitamin D-related compound(s) can be decreased relative to a reference level if the level of the vitamin D-related compound(s) is 85%, or less, 75% or less, 50% or less, 40% or less, 30% or less, 20% or less, or less than the reference level. In some embodiments, the level of a vitamin D-related compound(s) can be normalized relative to the level of one or more reference genes, reference proteins, or reference compounds. In some embodiments, the level of a vitamin D-related compound(s) can be normalized relative to a reference value.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of a given disease and/or deficiency. A subject can be male or female.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein “binding protein” refers to a protein which binds the analyte. The binding protein can be specific for the analyte, or can bind the analyte at a level and/or affinity which does not rise to specific binding. In some embodiments of any of the aspects, the binding protein binds specifically to the analyte.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “specific binding” refers to a chemical interaction between two molecules, compounds, cells and/or particles wherein the first entity binds to the second, target entity with greater specificity and affinity than it binds to a third entity which is a nontarget. In some embodiments, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A method of measuring the level of an analyte in a sample wherein a portion of the analyte in the sample is bound by at least one binding protein, the method comprising: heating the sample to a temperature at which the binding protein is denatured; and measuring the level of the analyte.
2. The method of paragraph 1, wherein the analyte is one or more of:
   25-hydroxyvitamin D3; 25-hydroxyvitamin D2; 1,25-dihydroxyvitamin D3; 1,25-dihydroxyvitamin D2; vitamin D3; vitamin D2 or a metabolite thereof.
3. The method of any of paragraphs 1-2, wherein the temperature is at least 40° C.
4. The method of any of paragraphs 1-3, wherein the temperature is from 40° C. to 250° C.
5. The method of any of paragraphs 1-4, wherein the temperature is at least 200° C.
6. The method of any of paragraphs 1-5, wherein the heating step comprises exposing the sample to at least one of:
   an electric heating device; an infrared signal; an electromagnetic signal; a microwave signal; or an exothermic chemical reaction.
7. The method of any of paragraphs 1-6, wherein the method further comprises a step after the heating step and before the measuring step of extracting the sample.
8. The method of any of paragraphs 1-7, wherein the method further comprises a step after the heating step and before the measuring step of extracting the sample with an organic solvent.
9. The method of any of paragraphs 1-8, wherein the measuring step comprises a chemiluminescence assay or radioimmunoassay.
10. The method of any of paragraphs 1-9, wherein the measuring step comprises liquid chromatography.
11. The method of any of paragraphs 1-10, wherein the measuring step comprises mass spectrometry.
12. The method of any of paragraphs 1-11, wherein the measuring step comprises measuring the level of vitamin D and/or a metabolite thereof.
13. The method of any of paragraphs 1-12, wherein the sample is a serum or plasma sample.
14. The method of any of paragraphs 1-12, wherein the sample is a saliva sample
15. The method of any of paragraphs 1-14, wherein the pH of the sample is no lower than 5.6 during the method.

### EXAMPLES

### Example 1

Patients are evaluated for their vitamin D status by measuring 25-hydroxyvitamin D. There are two forms: 25-hydroxyvitamin D3 and 25-hydroxyvitamin D2. Currently available assays are inaccurate, as at least a portion of the vitamin 25-hydroxyvitamin D2 is bound to proteins and thus unavailable for measurement.

In the methods described herein, a sample (e.g. a serum sample) is first denatured to cause the proteins in the sample to release 25-hydroxyvitamin D2 and 25-hydroxyvitamin D3. This sample can then extracted with an organic solvent and then after a few steps, both 25-hydroxyvitamin D2 and 25-hydroxyvitamin D3 levels are determined, e.g., by a chemiluminescence radioimmunoassay.

In some embodiments, the denaturing step can be a heat denaturation, e.g., heating the sample to at least 200° C. Heat denaturation can be performed using, e.g, an electric heating device, infrared technology within LED or other methods of quickly heating up a serum sample up to 200° C. to release quantitatively 25-hydroxyvitamin D2 and 25-hydroxyvitamin D3 from serum binding proteins.

The methods described herein can be utilized in, e.g, automated platforms and/or to quantitatively measure free vitamin D and its metabolites from the vitamin D binding protein and other proteins in body fluids especially in serum and plasma that results in the quantitative determination of the vitamin D and vitamin D metabolites. Of particular interest is the ability to quantitatively release 25-hydroxyvitamin D2 and 25-hydroxyvitamin D3 from its binding protein the vitamin D binding protein in the serum to permit the quantitative determination of both of these metabolites using the platform chemiluminescence assay.

In some embodiments, denaturation with an acid or base is not completely effective in releasing 25-hydroxyvitamin D2 from binding proteins making it unavailable for measurement. As a result, the assay can underestimates the total 25-hydroxyvitamin D in the circulation of a patient who is on vitamin D2 to treat and prevent vitamin D deficiency.

In some embodiments, the methods described herein can be applied to the measurement of other protein-bound analystes.

What is claimed herein is:

1. A method of measuring the level of at least one analyte in a sample, wherein the analyte is selected from the group consisting of:
25-hydroxyvitamin D3; 25-hydroxyvitamin D2; 1,25-dihydroxyvitamin D3; 1,25-dihydroxyvitamin D2; vitamin D3; vitamin D2; or a metabolite thereof
wherein a portion of the analyte in the sample is bound by at least one binding protein, the method comprising:
heating the sample to a temperature at which the binding protein is denatured; and
measuring the level of the analyte.

2. The method of claim 1, wherein the temperature is at least 40° C.

3. The method of claim 1, wherein the temperature is from 40° C. to 250° C.

4. The method of claim 1, wherein the temperature is at least 200° C.

5. The method of claim 1, wherein the heating step comprises exposing the sample to at least one of:
an electric heating device; an infrared signal; an electromagnetic signal; a microwave signal; or an exothermic chemical reaction.

6. The method of claim 1, wherein the method further comprises a step after the heating step and before the measuring step of extracting the sample.

7. The method of claim 6, wherein the extraction step comprises the use of an organic solvent.

8. The method of claim 1, wherein the measuring step comprises a chemiluminescence assay or radioimmunoassay.

9. The method of claim 1, wherein the measuring step comprises liquid chromatography.

10. The method of claim 1, wherein the measuring step comprises mass spectrometry.

11. The method of claim 1, wherein the sample is a serum or plasma sample.

12. The method of claim 1, wherein the sample is a saliva sample.

13. The method of claim 1, wherein the pH of the sample is no lower than 5.6 during the heating and measuring steps.
